(19) United States
(12) Patent Application Publication
(10) Pub. No.: US 2016/0022856 A1
(43) Pub. Date: Jan. 28, 2016

(54) SUPPORT AND HOLDING DEVICE FOR MELTABLE TARTS

(71) Applicant: Scott H. PRYNE, New Windsor, NY (US)

(72) Inventor: Scott H. PRYNE, New Windsor, NY (US)

(73) Assignee: AMERICAN FELT & FILTER COMPANY, New Windsor, NY (US)

(21) Appl. No.: 14/718,519

(22) Filed: May 21, 2015

Related U.S. Application Data
(60) Provisional application No. 62/028,024, filed on Jul. 23, 2014.

(51) Int. Cl. A61L 9/03

(52) U.S. Cl. CPC A61L 9/03

(57) ABSTRACT

A device and method for supporting a meltable tart and removing hot molten wax or other material from the tart when heated. The device has a heat resistant body formed of a material that is of sufficient porosity to absorb or soak up hot molten wax or other material from the tart and to retain its structural integrity so that it can be disposed of after it has absorbed or soaked up the hot molten wax or other material from the used tart.

### CROSS REFERENCE TO RELATED APPLICATIONS

The present application claims the priority of Provisional Patent Application No. 62/028,024 filed on Jul. 23, 2014.

### FIELD OF THE INVENTION

The present invention relates to a support and holding device for a tart formed of wax or other material and, more particularly, to such a device that has a porous and heat resistant body with a meltable tart mounted therein and when heated, absorbs or soaks up the hot molten wax or other material from the tart and can then be disposed of.

### DESCRIPTION OF THE PRIOR ART

Various devices are currently used to emit scents in a surrounding area. For example, scented candles may be burned or wax or candle tarts may be heated and melted to emit a scent. In the case of a candle, there is a safety hazard resulting from an open flame.

In the case of a meltable tart formed of wax or other material, it turns to liquid when heated and forms a pool that emits a fragrance. The pool is in the form of a hot liquid and thus may spill and result in a burn to the user or damage to a supporting surface. Additionally, it is a common practice to allow the hot liquid wax or other material from the candle tart to cool and thus harden in a container in which it is received. It is difficult and time consuming to attempt to remove the hardened wax or other material from the receptacle.

A need has arisen, therefore, for a new and improved device and method for collecting and disposing of hot molten wax or other material from a meltable tart or the like. The support and holding device and method of the present invention meet this need.

### BRIEF SUMMARY OF THE INVENTION

The support and holding device of the present invention has a body of any suitable size or shape and is formed of any suitable heat-resistant material that will absorb or soak up hot molten wax or other material. A scented meltable tart is mounted in the body of the device. In the method of the present invention, the body of the device, when heated, absorbs or soaks up the hot molten wax or other material from the tart that emits a scent. After use, the device can then be disposed of in a convenient manner.

### BRIEF DESCRIPTION OF THE DRAWINGS

FIG. 1 is a plan view of a first embodiment of the support and holding device of the present invention;

FIG. 2 is a sectional view taken substantially along line 2-2 of FIG. 1; and

FIG. 3 is a sectional view similar to FIG. 2 showing a second embodiment of the present invention.

### DETAILED DESCRIPTION OF THE INVENTION

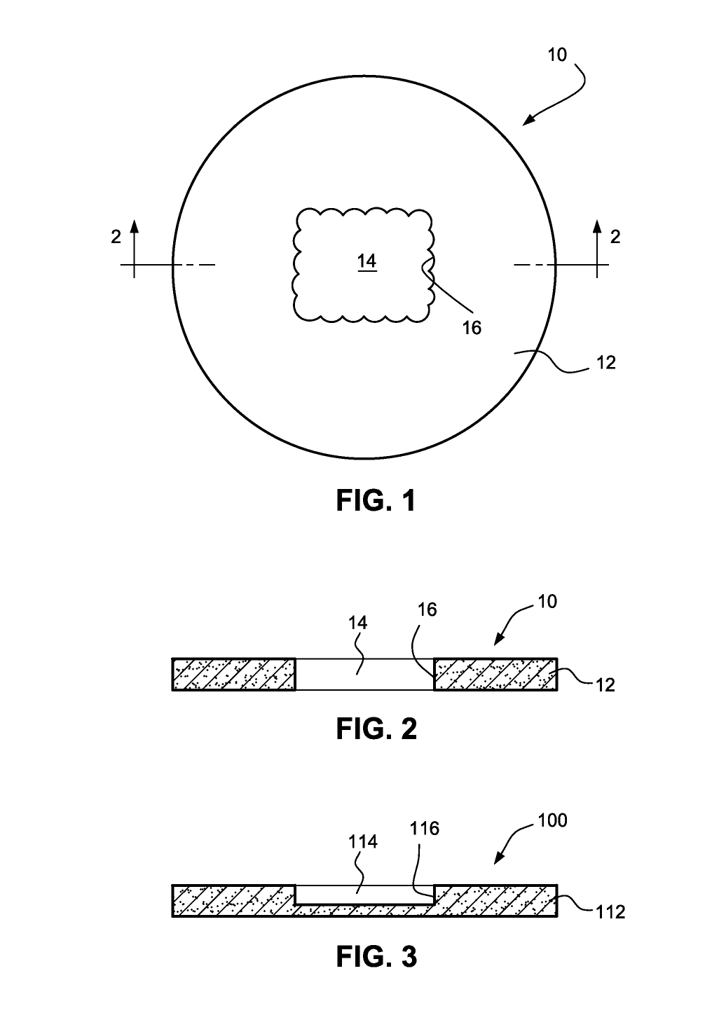

The support and holding device 10 of the present invention has a body 12 of any suitable size or shape and a scented meltable tart 14 formed of wax or another material of any suitable type is mounted in a center or other portion 16 thereof, as shown in FIGS. 1 and 2. Preferably, the shape of the portion 16 includes a large amount of surface area surrounding the tart 14 for absorbing or soaking up hot molten wax or other material from the tart. The body 12 is of any suitable shape such as the shape of a wafer or a similar shape.

The body 12 of the support and holding device 10 of the present invention is formed of any suitable porous and heat resistant material that will absorb or soak up hot molten wax or other material and maintain its structural integrity when heated. As illustrative examples, the body 12 may be formed of any nonwoven, woven, meltblown, spunbond, sintered, thermo bonded, extruded or resin bonded material comprised of any natural or synthetic blend and varied deniers of fibers, or of any suitable capillary wicking material. The density can be adjusted so that its porosity will be suitable for complete pick up of hot molten wax or other material from the tart 14 or the like.

In the method of the present invention, the device 10 is heated to melt the wax or other material of the tart 14 so that it emits a scent. The body 12 of the device 10 has sufficient porosity and heat resistance so that it absorbs or soaks up substantially all of the hot molten wax or other material from the tart 14 and maintains its structural integrity so that it can then be disposed of after use. In this manner, the hot molten wax or other material of the tart 14 can be easily disposed of without exposing the user to burns from the hot material or any damage to a supporting surface if the hot material should spill. Also, it is not necessary to wait for the holt molten wax or other material of the tart 14 to cool and harden which makes it very time consuming and difficult for removal of the hardened wax or other material from a receptacle for the tart 14 or the like.

FIG. 3 shows a second embodiment of a support and holding device 100 of the present invention having a body 112 with a recessed portion 116 in which the meltable tart 114 is mounted.

As will be evident from the foregoing description, the support and holding device and method of the present invention are advantageous in handling a scented meltable tart and in removing and disposing of the molten wax or other material from the tart or the like for many reasons, some of which are as follows:

1. The support and holding device is simple in construction and is inexpensive to manufacture;

2. The device is easy to store, handle and use;

3. The device allows for the immediate removal and disposal of hot molten wax or other material from a tart and thus minimizes the chance of burns to a user or damage to a supporting surface if the hot molten wax or other material is spilled;

4. The device is reliable in operation and absorbs or soaks up hot molten wax or other material from the tart mounted therein without requiring a user to wait for the hot molten material to cool and harden before it can be removed from a receptacle which is time consuming and difficult;

5. The device can be manufactured with a large surface area surrounding the tart to facilitate the absorption of the hot molten wax or other material; and

6. The device can be easily disposed of after use.

While the invention has been described in connection with what is presently considered to be the most practical and preferred embodiments, it is to be understood that the invention is not to be limited to the disclosed embodiments but on the contrary, is intended to cover various embodiments and equivalent arrangements included within the spirit and scope of the appended claims.

1. A device for supporting a meltable tart and disposing of hot molten wax or other material from the tart when heated, comprising:
a heat resistant body formed of a material that is of sufficient porosity to absorb or soak up hot molten wax or other material from a tart when melted and to retain structural integrity so that the device can be disposed of after it has absorbed or soaked up the hot wax or other material, and
a meltable scented tart mounted within a portion of the body.

2. The device of claim 1 wherein the body has a surface area surrounding the tart that is sufficient to absorb or soak up substantially all of the hot molten wax or other material from the tart when it is melted.

3. The device of claim 1 wherein the body is formed of a material selected from the group comprising nonwoven, woven, meltblown, spunbond, sintered, thermo bonded, extruded or resin bonded material.

4. The device of claim 3 wherein the material is comprised of a natural or synthetic blend and varied denier of fibers.

5. The device of claim 1 wherein the body has a density and porosity to absorb or soak up substantially all of the hot molten wax or other material from the tart when it is melted.

6. The device of claim 1 wherein the tart is mounted within a center portion of the body.

7. The device of claim 6 wherein the center portion is an aperture through the body.

8. The device of claim 6 wherein the center portion is a recess in the body.

9. A method of supporting a meltable scented tart and removing and disposing of hot molten wax or other material from the tart when heated and used, comprising:
mounting a meltable tart in a portion of a body of a support device formed of a heat-resistant material that absorbs or soaks up hot molten wax or other material from the tart when heated,
whereby when the tart is heated, the body retains structural integrity and can be disposed of when it has absorbed or soaked up the hot wax or other material from the used tart.

10. The method of claim 9 wherein the body has a size, shape and surface area surrounding the tart sufficient to absorb or soak up substantially all of the hot molten wax or other material from the tart.

11. The method of claim 10 wherein the body is formed of a material selected from the group comprising nonwoven, woven, meltblown, spunbond, thermo bonded, extruded or resin bonded material.

12. The method of claim 11 wherein the material is comprised of a natural or synthetic blend and varied denier of fibers.

13. The method of claim 9 wherein the meltable tart is mounted within a center portion of the body of the support device.

14. The method of claim 13 wherein the center portion is an aperture through the body.

15. The method of claim 13 wherein the center portion is a recess in the body.
